(19) United States
(12) Patent Application Publication
(10) Pub. No.: US 2025/0339951 A1
(43) Pub. Date: Nov. 6, 2025

(54) ARM SUPPORT SYSTEMS AND METHODS FOR USING THEM

(71) Applicant: Levitate Technologies, Inc., Del Mar, CA (US)

(72) Inventors: Mark C. Doyle, Del Mar, CA (US); David A. Mansfield, San Diego, CA (US)

(21) Appl. No.: 18/654,990

(22) Filed: May 3, 2024

Related U.S. Application Data
(63) Continuation of application No. PCT/US22/48897, filed on Nov. 3, 2022.
(60) Provisional application No. 63/275,332, filed on Nov. 3, 2021.

(51) Int. Cl. B25J 9/00; A61F 5/01; B25J 19/00

(52) U.S. Cl. CPC B25J 9/0006; A61F 5/013; B25J 19/0016; A61F 2005/0197; A61H 2201/1635

(57) ABSTRACT

An arm support system includes an arm support coupled to a connection bracket about a pivot joint and carrying an arm rest for receiving a user's upper arm. Compensation elements are coupled to the arm support to apply an offset force to at least partially offset a gravitational force acting on the arm as the user raises and lowers the arm including a spring, a cable coupled between a connection post on the connection bracket and the spring, thereby defining an axis along a segment of the cable, one or more pulleys mounted on the arm support such that the cable wraps at least partially around the pulleys to modify the offset force as the user raises and lowers the arm, and a feature mounted adjacent the connection post that modifies the cable axis relative to the pivot joint to reduce the offset force as the arm support is lowered.

### RELATED APPLICATION DATA

The present application is a continuation of International Application No. PCT/US2022/048897, filed Nov. 3, 2022, which claims benefit of U.S. provisional application Ser. No. 63/275,332, filed Nov. 3, 2021, the entire disclosures of which are expressly incorporated by reference herein.

### TECHNICAL FIELD

The present application relates to systems, devices, and methods for supporting a user's arms, for example, to adaptive arm support systems that support one or both of a user's arms, while allowing substantially free motion, e.g., to allow the user to perform one or more tasks for extended periods of time with one or both arms extended.

### BACKGROUND

A single-cable arm support mechanism has been described in U.S. Pat. No. 9,737,374, the entire disclosure of which is expressly incorporated by reference herein. This arm support mechanism varies the restoring moment that it applies to a user's arm, thereby counterbalancing the weight of the user's arm in a way that is adaptive to the position of the arm. The restoring moment increases and decreases on an approximately sinusoidal basis in response to the angle of the user's arm. For example, the restoring moment is maximized when the user's arm is horizontal because the center of mass of the arm is farthest from the user's shoulder, and thus the gravitational force acting on the arm has the greatest influence. When the user lowers their arm, e.g., towards a vertical or relaxed position, the distance of the center of mass from their shoulder decreases trigonometrically, and the gravitational force on the arm has reduced influence. The arm support mechanism referenced above uses simple trigonometric relationships to reduce the moment applied to the arm when the user lowers it. Many users find that the reduction in moment as they lower their arm is insufficient, because the restoring moment, following trigonometric relationships, is not diminishing rapidly enough.

Thus, there is a need for arm support mechanisms and systems that that have an accelerated reduction in the restoring moment as the user lowers their arm.

### SUMMARY

The present application is directed to systems, devices, and methods for supporting a user's arms, for example, to adaptive arm support systems that support one or both of a user's arms, while allowing substantially free motion, e.g., to allow the user to perform one or more tasks for extended periods of time with one or both arms extended.

In accordance with one example, a system is provided for supporting an arm of a user that includes a harness configured to be worn on a body of a user; an arm support coupled to the harness comprising a first end coupled to a connection bracket at a pivot joint and a second end comprising an arm rest for receiving an upper arm of the user, the arm support configured to pivot about the pivot joint to accommodate and follow the user raising and lower the arm; and one or more compensation elements coupled to the arm support to apply an offset force to at least partially offset a gravitational force acting on the arm as the user raises and lowers the arm. The one or more compensation elements may include a spring including a first end mounted to the arm support; an elongate tension element, e.g., cable, comprising a first end coupled to a connection post on the connection bracket and a second end coupled to a second end of the spring, thereby defining an axis extending along a segment of the tension element from the second end of the arm support towards the first end of the arm support. One or more pulleys are mounted on the arm support such that the tension element wraps at least partially around the one or more pulleys to modify the offset force as the user raises and lowers the arm, and a feature is mounted adjacent the connection post such that, when the arm support is rotated about the pivot joint above a predetermined angle, the feature offsets the axis away from the pivot joint and, when the arm support is rotated below the predetermined angle, the axis moves closer to the pivot joint to reduce the offset force.

In another example, a system is provided for supporting an arm of a user that includes a harness configured to be worn on a body of a user; an arm support coupled to the harness comprising a first end coupled to a connection bracket at a pivot joint and a second end comprising an arm rest for receiving an upper arm of the user, the arm support configured to pivot about the pivot joint to accommodate and follow the user raising and lower the arm; and one or more compensation elements coupled to the arm support to apply an offset force to at least partially offset a gravitational force acting on the arm as the user raises and lowers the arm. The one or more compensation elements may include a spring including a first end mounted to the arm support; an elongate tension element comprising a first end coupled to a connection post on the connection bracket and a second end coupled to a second end of the spring, thereby defining an axis extending along a segment of the tension element from the second end of the arm support towards the first end of the arm support; a first pulley mounted to the arm support adjacent the second end of the arm support; a second pulley mounted to the arm support adjacent the connection bracket and wherein the tension element extends from the second end of the spring, partially around the first pulley, and partially around the second pulley to modify the offset force as the arm support rotates about the pivot joint; and a feature mounted adjacent the connection post such that, when the arm support is rotated about the pivot joint above a predetermined angle, the feature offsets the axis away from the pivot joint and, when the arm support is rotated below the predetermined angle, the axis moves closer to the pivot joint to reduce the offset force.

In accordance with still another example, a system is provided for supporting an arm of a user that includes a harness configured to be worn on a body of a user; an arm support coupled to the harness comprising a first end coupled to a connection bracket at a pivot joint and a second end comprising an arm rest for receiving an upper arm of the user, the arm support configured to pivot about the pivot joint to accommodate and follow the user raising and lower the arm; and one or more compensation elements coupled to the arm support to apply an offset force to at least partially offset a gravitational force acting on the arm as the user raises and lowers the arm. The one or more compensation elements may include a spring including a first end mounted to the arm support; an elongate tension element comprising a first end coupled to a connection post on the connection bracket and a second end coupled to a second end of the spring, thereby defining an axis extending along a segment of the tension element from the second end of the arm support towards the first end of the arm support; a first pulley mounted to the arm support adjacent the second end of the arm support; a second pulley mounted to the arm support adjacent the connection bracket and wherein the tension element extends from the second end of the spring, partially around the first pulley, and partially around the second pulley to modify the offset force as the arm support rotates about the pivot joint; and a wrap feature mounted on the connection bracket adjacent the connection post such that, when the arm support is rotated about the pivot joint above a predetermined angle, the tension element wraps partially around the wrap feature and, when the arm support is rotated below the predetermined angle, the wrap feature is released from wrapping around the wrap feature to reduce the offset force as the arm support moves towards a substantially vertically downward orientation.

In yet another example, a system is provided for supporting an arm of a user that includes a harness configured to be worn on a body of a user; an arm support coupled to the harness comprising a first end coupled to a connection bracket at a pivot joint and a second end comprising an arm rest for receiving an upper arm of the user, the arm support configured to pivot about the pivot joint to accommodate and follow the user raising and lower the arm; and one or more compensation elements coupled to the arm support to apply an offset force to at least partially offset a gravitational force acting on the arm as the user raises and lowers the arm. The one or more compensation elements may include a spring including a first end mounted to the arm support; an elongate tension element comprising a first end coupled to a connection post on the connection bracket and a second end coupled to a second end of the spring, thereby defining an axis extending along a segment of the tension element from the second end of the arm support towards the first end of the arm support; a first pulley mounted to the arm support adjacent the second end of the arm support; and a second pulley mounted to the arm support adjacent the connection bracket and wherein the tension element extends from the second end of the spring, partially around the first pulley, and partially around the second pulley to modify the offset force as the arm support rotates about the pivot joint; wherein the connection post comprises a pivoting cable link, and wherein one or more pivot features are mounted on the connection bracket and coupled to the cable link to allow the cable link to translate relative to the pivot joint when the arm support is rotated below the predetermined angle to reduce the offset force as the arm support moves towards a substantially vertically downward orientation.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIGS. 1A-1C show an example of an arm support carrying an arm rest and compensation elements for supporting a user's arm.

FIG. 1D shows a graph of an exemplary restoring moment provide provided by two support mechanisms, e.g., a first solid line representing a restoring moment that may be applied by the arm support mechanism shown in FIGS. 1A-1C and a dashed line showing a more desired restoring moment, at various angles of the user's arm.

FIGS. 2A-2C show another example of an arm support carrying an arm rest and compensation elements for supporting a user's arm.

FIGS. 3A-3C show yet another example of an arm support carrying an arm rest and compensation elements for supporting a user's arm.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

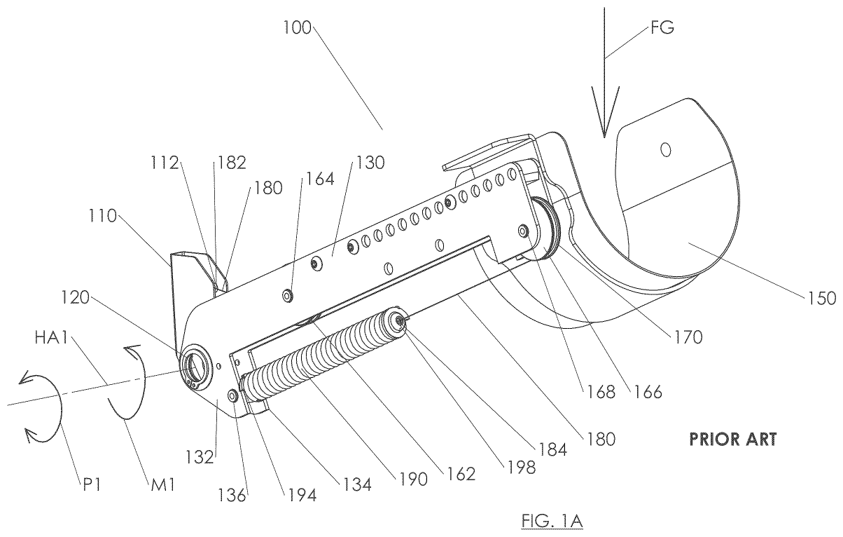

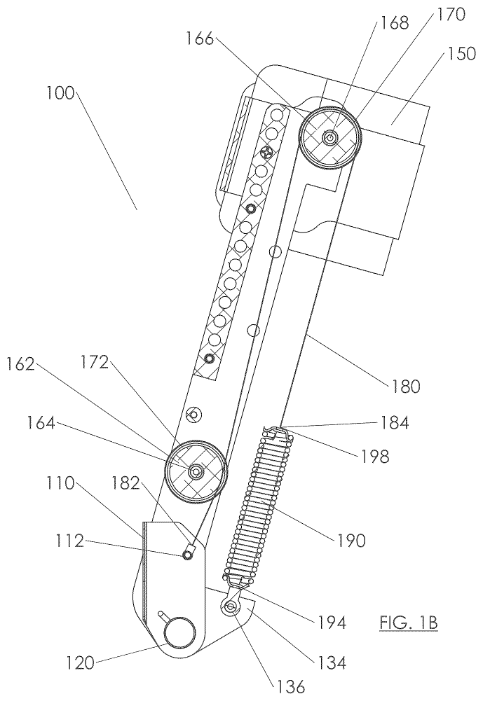

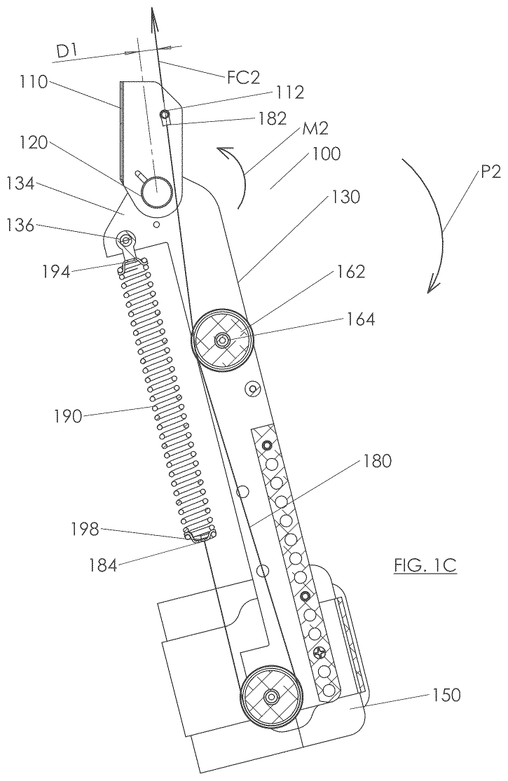

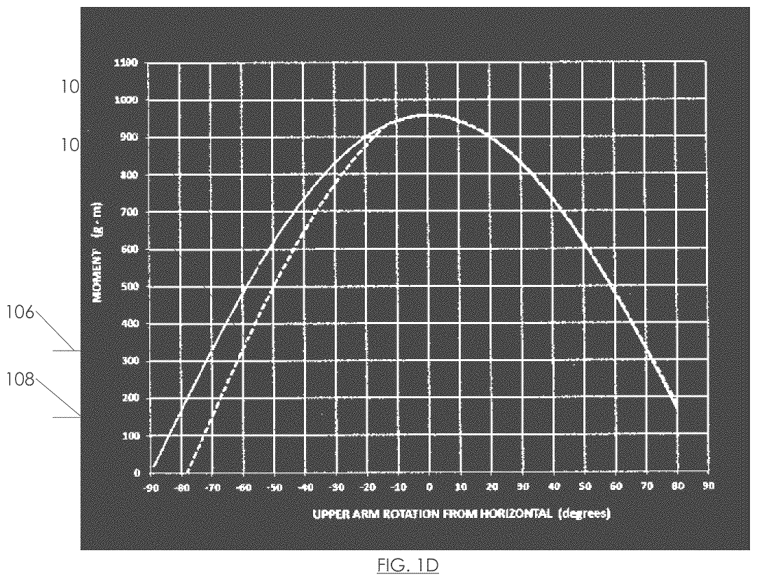

Turning to the drawings, FIG. 1A shows a perspective view of an arm support mechanism 100 that may be attached to a harness (not shown) worn on the torso of a user or other structure at connection bracket 110, e.g., to provide an arm support system that may be used to support one or both arms of a user, e.g., while working or otherwise engaged in activities with the supported arm(s) extended for an extended period of time. A connection bracket 110 and support arm 130 form a pivot joint 120, e.g., at a first end of the support arm 130, defining a generally horizontal axis HA1, about which support arm 130 can pivot approximately along path P1. An armrest 150 is attached to support arm 130, e.g., mounted on a second end of the support arm 130 opposite the connection bracket 130, and accepts the upper arm of the user (not shown). The support arm 130 may be constructed from one or more side plates or other mounting structures, e.g., plates 132, 134, which provide mounting for pulleys 162 and 166, which may pivot about shafts 164 and 168 respectively. Alternatively, other elongate segment members, e.g., one or more brackets, shafts, and the like (not shown) may be provided that extend from a first end of the mechanism 100 to a second end supporting the upper arm.

A cable 180 includes a proximal end 182 attached to connection post 112 of the connection bracket 110, and includes a distal end 184 attached to a distal end 198 of spring 190. A proximal end 194 of the spring 190 is attached to the support arm 130 at spring connection feature 136. The cable 180 wraps around pulleys 162 and 168, e.g., in pulley groove(s) 170. While the descriptions herein refer to cables, it is understood that other tension elements, e.g., one or more belts, chains, cords, and the like, may be substituted for the cable 180.

The weight of the user's arm and any tool held by the user applies a gravitational force FG to the armrest 150 receiving the upper arm, creating a moment about the pivot joint 120. The arm support mechanism 100 applies a restoring moment M1 to counterbalance all of, or a portion of, the moment created by the gravitational force FG about the pivot joint 120.

FIG. 1B shows a side section view of the arm support mechanism 100 of FIG. 1A. As shown, the arm support mechanism 100 is in an arm-raised position, consistent with the user raising their arm. The proximal end 182 of the cable 180, attached to the connection post 112 of the connection bracket 110, is wrapped around pulleys 162, 166. The distal end 184 of the cable 180 is attached to the distal end 198 of the spring 190. The proximal end 194 of the spring 190 is attached to the support arm 130 at the connection feature 136. As shown, in this elevated position, the spring 190 is providing a relatively low tension to the cable 180.

FIG. 1C shows a side section view of the arm support mechanism 100 of FIGS. 1A and 1B in an arm-lowered position, having rotated about the pivot joint 120 approximately along path P2. As shown, the spring 190 is distended, and is providing a relatively large tension FC2 to cable 180. Tension FC2, acting on the connection post 112 of the connection bracket 110, acts at a small offset distance D1 from the center of rotation of the pivot joint 120, and thereby applies a potentially significant moment M2 about the pivot joint 120. In some cases, users perceive this moment as excessive.

FIG. 1D shows a graph of exemplary restoring moment profiles provided by two support mechanisms at various angles of the user's arm, e.g., with zero degrees (0°) corresponding to the user's upper arm being oriented horizontally. The arm support mechanism of FIGS. 1A-1C may follow curve 102, which may be a generally sinusoidal curve, and which may provide maximum restoring moment at zero degrees, consistent with the user's upper arm being substantially horizontal. Curve 102 shows that the restoring moment provided by the arm support mechanism 100 does diminish as the user lowers their arm. For example, the restoring moment at negative forty degrees (−40°), i.e., below horizontal, is less than that at zero degrees (horizontal). However, some users find that the restoring moment as they lower their arm is still uncomfortably high and may interfere with free movement.

A more ideal restoring moment curve would have a more rapid reduction as the user lowers their arm below horizontal, for example, as represented by curve 104 in FIG. 1D. For example, at an arm position of negative seventy degrees (−70°), i.e., seventy degrees below horizontal, an arm support mechanism following curve 102 would provide a restoring moment of 330 g-m, while one following curve 104 would provide 150 g-m, a significant difference, that may increase comfort for a user. The curve 104 is provided as an illustrative example and it will be appreciated that other force reduction curves are contemplated and may be provided in arm support mechanisms, as desired.

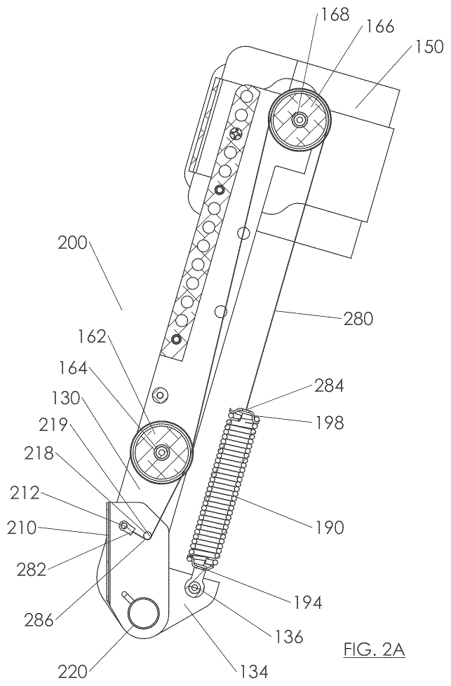

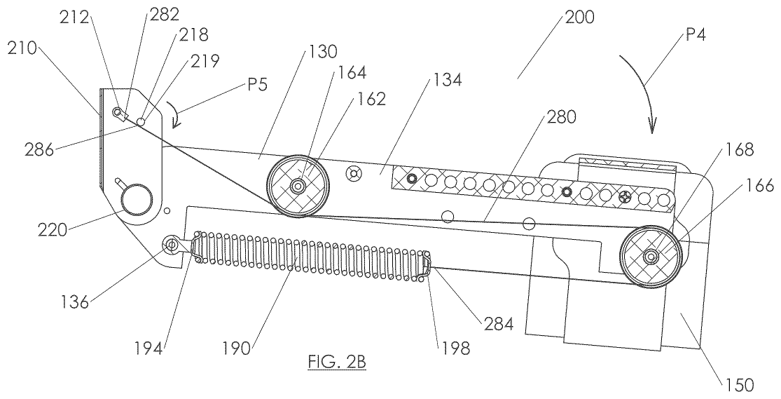

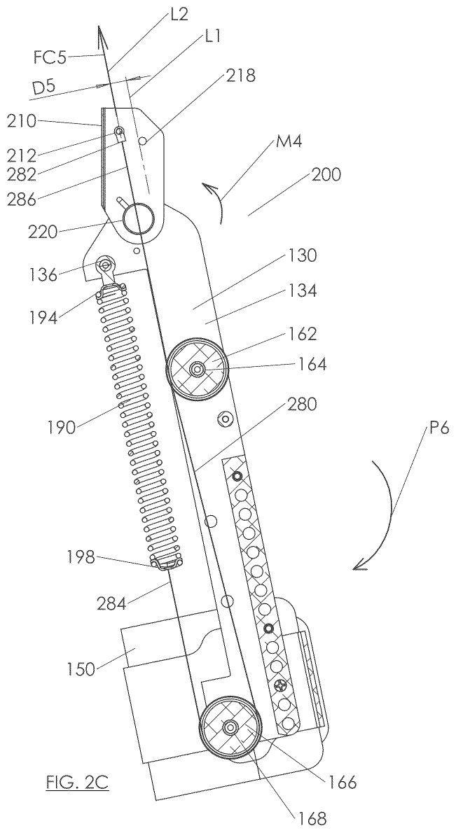

Turning to FIGS. 2A-2C, side views of an example of an improved arm support mechanism 200 is shown, which may provide a greater-reduced restoring moment as the user lowers the arm, e.g., as shown in FIG. 2C. The arm support mechanism 200 may be included in exoskeletons or other arm support systems, such as those disclosed in U.S. Pat. Nos. 9,205,017, 9,737,374, and U.S. Publication No. 2018/0303650, the entire disclosures of which are expressly incorporated by reference herein.

As shown, the arm support mechanism 200 includes a connection bracket 210, a support arm 130 connected at pivot joint 220 at a first end of the support arm 130 and an armrest 150 mounted on a second end of the support arm 130, generally similar to the mechanism 100. The connection bracket 210 may be connected to other components of an arm support system, e.g., a shoulder bracket that is coupled a harness of the arm support system (not shown), which may pivot about a vertical axis above a user's shoulder when the harness is worn, as described further in the references incorporated above. For example, the arm support mechanism 200 may be connected to a lower end of such a shoulder bracket to follow movement of the user's upper arm received in the armrest 150, e.g., as the user raises and lowers the upper arm, as described further elsewhere herein and in the references incorporated above. The connection bracket 210 may be removable from an arm support system, e.g., including one or more cooperating connectors on the connection bracket 210 and shoulder bracket (not shown) or the connection bracket 210 may be permanently connected to an arm support system.

Optionally, the arm support mechanism 200 may include one or more additional features, e.g., one or more covers to contain the components, one or more straps to secure a user's arm in the armrest, and the like (not shown). The harness may include one more components for securing the arm support system to the user's body, e.g., a pair of shoulder straps that may be received over the user's shoulder (e.g., to which a shoulder bracket may be attached), a belt, pad, and/or other structure that may be secured around the user's hips, waist, and/or other location on the torso, and one or more support tubes, struts, and/or other rigid structures for transferring forces from the arm support mechanism 200 to other locations on the user's body.

As shown in FIG. 1A, a proximal end 282 of cable 280 is attached at connection post 212 of the connection bracket 210. A distal end 284 of the cable 280 is attached to a distal end 198 of a spring 190. A proximal end 194 of the spring 190 is attached to the support arm 130 at connection feature 136, e.g., adjacent the first end of the arm support mechanism 200. As shown in FIG. 1A, the spring 190 is providing a relatively low tension to the cable 280 when the arm is raised above horizontal. An intermediate portion 286 of the cable 280 is shown wrapped around a contact surface 219 of a wrap feature 218.

In the example shown, the wrap feature 218 is a post mounted on the connection bracket 210 adjacent the connection post 212. The wrap feature 218 may be spaced apart and/or offset from the connection post 212, e.g., diagonally below the connection post 212, to allow the cable 280 to contact and/or be released from contacting the wrap feature 218 as the arm support mechanism 200 rotates, as described further elsewhere herein. The contact surface 219 may extend at least partially around the wrap feature 218 and may have one or more of a variety of shapes, including circular, sharp, square, straight, of multi curvatures, a spline, or many other advantageous shapes and sizes, e.g., to transfer the tension force from the spring 190 while allowing the cable 280 to slide around the wrap feature 218. As in the previous mechanism 100, the cable 280 also wraps partially around pulleys 162, 166, e.g. to transfer the spring force from the spring 190 and/or modify the moment that is applied to the arm support mechanism 200 as the user raises and lowers the supported arm.

FIG. 2B shows a side section view of the improved arm support mechanism 200 of FIG. 2A, shown in an arm-partially-lowered position, having been lowered approximately along path P4, e.g., to a substantially horizontal orientation. As the support mechanism 200 is lowered, the intermediate portion 286 of the cable 280 unwraps from the contact surface 219 of the wrap feature 218, approximately along path P5. For example, at the arm position shown in FIG. 2B, the cable intermediate portion 286 is about to separate from the contact surface 219 such that, during further lower rotation, the cable 286 no longer contacts the wrap feature 218.

FIG. 2C shows a side section view of the improved arm support mechanism 200 of FIG. 2B, shown in an arm-lowered position, having been lowered approximately along path P6 towards a substantially vertical orientation, e.g., corresponding to an orientation in which the user has lowered the arm to relax. As the support mechanism 200 has been lowered, the cable intermediate portion 286 has fully unwrapped from the contact surface 219 of the wrap feature 218, and the cable 280 is now directly in line with the connection post 212 of the connection bracket 210.

The line of action of the force FC5 in the cable 280 has shifted toward the pivot joint 220, and is now acting approximately along axis or line L2, instead of acting approximately along line L1, as it would be if the cable 280 was attached at the wrap feature 218. The line of action of the cable 280, and hence the tension FC5 in the cable 280, is now approximately distance D5 from the contact surface 219 of the wrap feature 218. The force FC5 in the cable 280, now acting closer to the center of the pivot joint 220 than would have been the case in other configurations, such as that shown in FIGS. 1A-1C, applies only a relatively insignificant restoring moment M4 about the pivot joint 220.

The result of allowing the cable intermediate portion 286 to unwrap from the contact surface 219 of the wrap feature 218 (at the arm elevation at which an increased diminishment of the restoring moment is desired) is in essence to have two different attachment points for the cable 280, one engaged when the user's arm is essentially horizontal and above (i.e., the contact surface 219), and the one engaged when the arm is lowered below horizontal (i.e., the connection post 212). The resulting effect is represented approximately by curve 104 of FIG. 1D.

The improved arm support mechanism 200 of FIGS. 2A-2C may achieve the desired effect of increased diminishment of the restoring moment as the user's arm is lowered. However, in order to be compact, the contact surface 219 may need to have a relatively small radius, which can shorten the life of tension elements such as cables or belts that need to wrap around it.

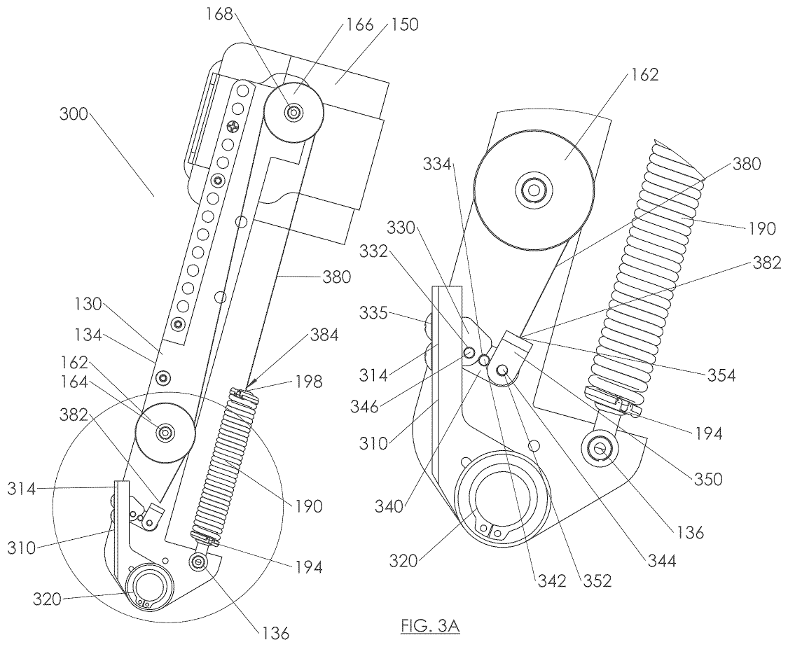

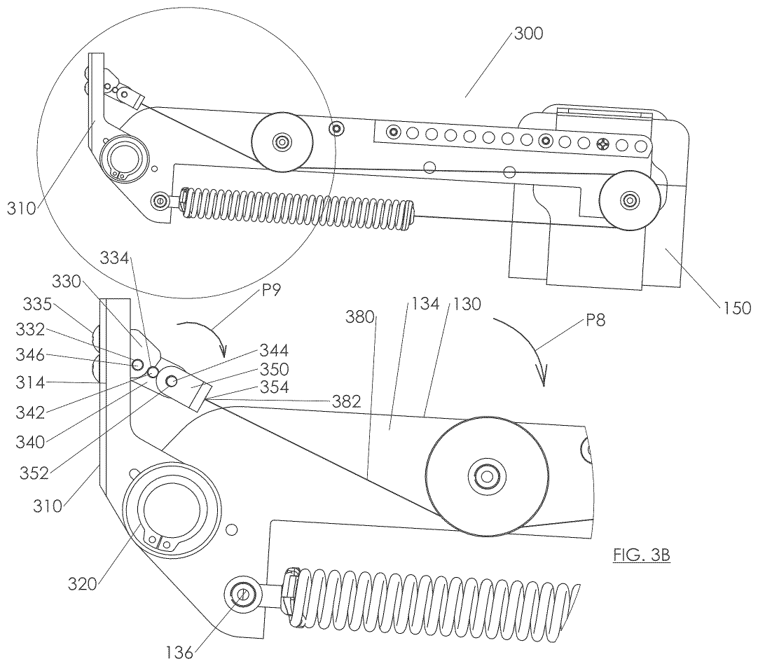

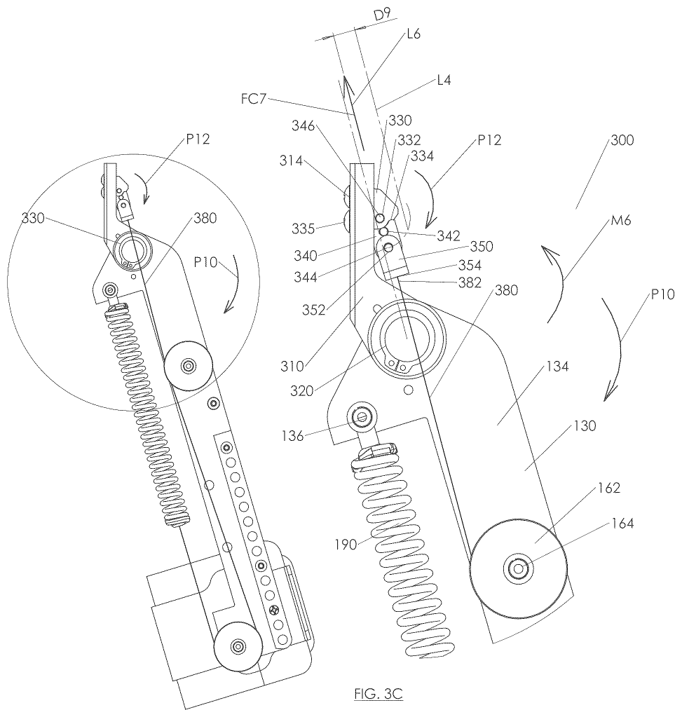

The exemplary arm support mechanism 300 shown in FIGS. 3A-3C may include some similar features to mechanism 200, as well as additional features to address this potential shortcoming. FIG. 3A shows a side view of the arm support mechanism 300, shown in an arm-raised position, with a side plate or other cover not shown for clarity to allow the internal components to be observed. A connection bracket 310 (which may be connected to a shoulder bracket or other components of an arm support system, not shown) and support arm 130 form a pivot joint 320, e.g., similar to the previous mechanisms. A proximal end 382 of cable 380 is attached at cable link end 354 of pivoting cable link 350, and a distal end 384 of the cable 380 is attached to a distal end 198 of spring 190. A proximal end 194 of the spring 190 is attached to the support arm 130 at connection feature 136.

As shown, the spring 190 is providing a relatively low tension to the cable 380. An upper bracket portion 314 of the connection bracket 310 provides a mounting point for a link anchor 330, which may be attached to the upper bracket portion 314, e.g., using one or more fasteners 335 and/or by any other suitable method. A pivot feature 332 of link anchor 330 rotates around first link shaft 346 of a link 340 to provide a pivot for the link 340, which may rotate about the pivot feature 332, e.g., as described further below. The link anchor 330 also provides a stop surface 334, which slidably engages with a link stop pin 342 on the link 340 to limit the upward rotation of the link 340. The link 340 further provides a second link shaft 344 about which a pivot feature 352 in a cable link 350 may rotate to enable rotation of the cable link 350 about the pivot feature 352.

FIG. 3B shows a side view of the arm support mechanism 300 of FIG. 3A, shown in an arm-partially-lowered position, e.g., oriented substantially horizontally, having been lowered approximately along path P8 (again with the side plate not shown for clarity). In response to the rotation of the arm support mechanism 300 about the pivot joint 320, the cable link 350 has rotated about the pivot feature 352, approximately along path P9. Although the cable link 350 has rotated about the pivot feature 352, the link 340 has not yet rotated about the pivot feature 332 of the link anchor 330 in this arm position. The stop pin 342 on the link 340 is still in contact with the stop surface 334 of the link anchor 330. The cable 380, attached at the cable link end 354 of the pivoting cable link 350, rotates about the pivot feature 352 in the cable link 350. To the user, the arm support mechanism 300 feels like it has a cable attached at the pivot feature 352 in the cable link 350.

FIG. 3C shows a side view of the arm support mechanism 300 of FIGS. 3A and 3B, shown in an arm-lowered position, e.g., substantially vertically downward, having been lowered approximately along path P10 (again with the side plate not shown for clarity). In response to the rotation of the arm support mechanism 300 about the pivot joint 320, the stop pin 342 on the link 340 has separated from the stop surface 334 of the link anchor 330, and the link 340 has rotated about the pivot feature 332 of the link anchor 330, approximately along path P12. As a result, the line of action of the cable 380, and hence the force FC7 in the cable 380, has moved closer to the center of the pivot joint 320. The line of action of the force FC7 in the cable 380, having shifted toward the pivot joint 320, is now acting approximately along axis or line L6, instead of acting along line L4, as it would be if the link 340 had not rotated about the pivot feature 332 of the link anchor 330. Thus, the force FC7 in the cable 380 is now acting approximately distance D9 from line L4. The force FC7 in the cable 380, acting now closer to the center of the pivot joint 320 than would have been the case in the configuration of the mechanism 100, can apply only a relatively insignificant restoring moment M6 about the pivot joint 320. To the user, the arm support mechanism 300 feels like it has a cable attached at the pivot feature 352 in the cable link 350 when their arm is above horizontal, and at the pivot feature 332 when their arm is below horizontal. The resulting effect is represented approximately by the curve 104 of FIG. 1D.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A system for supporting an arm of a user, comprising:
a harness configured to be worn on a body of a user;
an arm support coupled to the harness comprising a first end coupled to a connection bracket at a pivot joint and a second end comprising an arm rest for receiving an upper arm of the user, the arm support configured to pivot about the pivot joint to accommodate and follow the user raising and lower the arm;
one or more compensation elements coupled to the arm support to apply an offset force to at least partially offset a gravitational force acting on the arm as the user raises and lowers the arm, the one or more compensation elements comprising:
a spring including a first end mounted to the arm support;
an elongate tension element comprising a first end coupled to a connection post on the connection bracket and a second end coupled to a second end of the spring, thereby defining an axis extending along a segment of the tension element from the second end of the arm support towards the first end of the arm support;
one or more pulleys mounted on the arm support such that the tension element wraps at least partially around the one or more pulleys to modify the offset force as the user raises and lowers the arm; and
a feature mounted adjacent the connection post such that, when the arm support is rotated about the pivot joint above a predetermined angle, the feature offsets the axis away from the pivot joint and, when the arm support is rotated below the predetermined angle, the axis moves closer to the pivot joint to reduce the offset force.

2. The system of claim 1, wherein the axis intersects the pivot joint when the arm support is oriented substantially vertically downward.

3. The system of claim 1, wherein the arm support comprises one or more plates extending between the first and second ends of the arm support, and wherein the one or more pulleys are mounted to the one or more plates.

4. The system of claim 3, further comprising a cover mounted to the one or more plates to at least partially contain the spring and the tension element.

5. The system of claim 1, wherein the one or more pulleys comprise a first pulley mounted to the arm support adjacent the second end of the arm support and wherein the tension element extends partially around the first pulley.

6. The system of claim 5, further comprising a second pulley mounted to the arm support adjacent the connection bracket and wherein the tension element extends partially around the second pulley to modify the offset force as the arm support rotates about the pivot joint.

7. The system of claim 6, wherein the axis is aligned along a segment of the tension element extending between the second pulley towards the second end of the arm support.

8. The system of claim 5, wherein the first end of the spring is mounted to the arm support adjacent the first end of the arm support and the tension element extends from the second end of the spring around the first pulley and back towards the first end of the arm support to the connection post.

9. The system of claim 1, wherein the arm support is configured to extend partially along an upper arm of the user, and wherein the system further comprises a shoulder bracket coupled to the harness adjacent the shoulder of the user and to the connection bracket such that the shoulder bracket rotates about a vertical pivot joint.

10. The system of claim 9, wherein the shoulder bracket comprises a first end mounted to the harness above the user's shoulder and a second end coupled to the connection bracket.

11. The system of claim 10, wherein the connection bracket is removably connectable to the second end of the shoulder bracket.

12. The system of claim 10, wherein the connection bracket is permanently connected to the second end of the shoulder bracket.

13. The system of claim 1, wherein the predetermined angle is substantially horizontal.

14. The system of claim 1, wherein the connection post is pivotably mounted to the connector bracket to allow the tension element to extend from the connection bracket towards the second end of the arm support.

15. The system of claim 1, wherein the feature comprise a wrap feature mounted on the connection bracket adjacent the connection post such that the cable wraps partially around the wrap feature when the arm support is rotated above the predetermined angle.

16. The system of claim 15, wherein the tension element is released from wrapping around wrap feature when the arm support is rotated below the predetermined angle.

17. The system of claim 16, wherein a segment of the tension element is aligned with the connection post when the arm support is rotated below the predetermined angle.

18. The system of claim 1, wherein the connection post comprises a pivoting cable link, and wherein the feature comprises one or more pivot features mounted on the connection bracket and coupled to the cable link to allow the cable link to translate relative to the pivot joint when the arm support is rotated below the predetermined angle such that the axis moves closer to the pivot joint to reduce the offset force.

19-20. (canceled)

21. A system for supporting an arm of a user, comprising:
a harness configured to be worn on a body of a user;
an arm support coupled to the harness comprising a first end coupled to a connection bracket at a pivot joint and a second end comprising an arm rest for receiving an upper arm of the user, the arm support configured to pivot about the pivot joint to accommodate and follow the user raising and lower the arm;
one or more compensation elements coupled to the arm support to apply an offset force to at least partially offset a gravitational force acting on the arm as the user raises and lowers the arm, the one or more compensation elements comprising:
a spring including a first end mounted to the arm support;
an elongate tension element comprising a first end coupled to a connection post on the connection bracket and a second end coupled to a second end of the spring, thereby defining an axis extending along a segment of the tension element from the second end of the arm support towards the first end of the arm support;
a first pulley mounted to the arm support adjacent the second end of the arm support;
a second pulley mounted to the arm support adjacent the connection bracket and wherein the tension element extends from the second end of the spring, partially around the first pulley, and partially around the second pulley to modify the offset force as the arm support rotates about the pivot joint; and
a feature mounted adjacent the connection post such that, when the arm support is rotated about the pivot joint above a predetermined angle, the feature offsets the axis away from the pivot joint and, when the arm support is rotated below the predetermined angle, the axis moves closer to the pivot joint to reduce the offset force.

22-30. (canceled)

31. A system for supporting an arm of a user, comprising:
a harness configured to be worn on a body of a user;
an arm support coupled to the harness comprising a first end coupled to a connection bracket at a pivot joint and a second end comprising an arm rest for receiving an upper arm of the user, the arm support configured to pivot about the pivot joint to accommodate and follow the user raising and lower the arm;
one or more compensation elements coupled to the arm support to apply an offset force to at least partially offset a gravitational force acting on the arm as the user raises and lowers the arm, the one or more compensation elements comprising:
a spring including a first end mounted to the arm support;
an elongate tension element comprising a first end coupled to a connection post on the connection bracket and a second end coupled to a second end of the spring, thereby defining an axis extending along a segment of the tension element from the second end of the arm support towards the first end of the arm support;
a first pulley mounted to the arm support adjacent the second end of the arm support; and
a second pulley mounted to the arm support adjacent the connection bracket and wherein the tension element extends from the second end of the spring, partially around the first pulley, and partially around the second pulley to modify the offset force as the arm support rotates about the pivot joint;
wherein the connection post comprises a pivoting cable link, and wherein one or more pivot features are mounted on the connection bracket and coupled to the cable link to allow the cable link to translate relative to the pivot joint when the arm support is rotated below the predetermined angle to reduce the offset force as the arm support moves towards a substantially vertically downward orientation.

32. (canceled)
